(19) United States
(12) Patent Application Publication
(10) Pub. No.: US 2026/0078343 A1
(43) Pub. Date: Mar. 19, 2026

(54) Engineered Cell and Application Thereof

(71) Applicants: Tianhai Yuanqi Biotechnology (Tianjin) Co., Ltd., Tianjin City (CN); Hematology Hospital, Chinese Academy of Medical Sciences (Institute of Hematology, Chinese Academy, Tianjin (CN); Hebei Xiong'an Yuanqi Biotechnology Co., Ltd., Hebei Province (CN)

(72) Inventors: Tao CHENG, Tianjin (CN); Linping HU, Tianjin (CN); Sha HAO, Tianjin (CN); Shuquan RAO, Tianjin (CN); Yiwen HAN, Tianjin (CN)

(21) Appl. No.: 19/234,349

(22) Filed: Jun. 11, 2025

(30) Foreign Application Priority Data
Jun. 17, 2024 (CN) 202410772710.X

(51) Int. Cl. C12N 5/0789; C12N 5/10; C12N 9/22; C12N 15/113

(52) U.S. Cl. CPC C12N 5/0647; C12N 5/10; C12N 9/226; C12N 15/113; C12N 2310/20; C12N 2510/00

(57) ABSTRACT

The present invention provides an engineered cell and application thereof. Compared with a wild type cell, the engineered cell lacks or reduces the expression of HLA-A protein and HLA-B protein. The engineered cell disclosed by the present invention can significantly reduce immune rejection response after human hematopoietic cell infusion, and the implantation rate and long-term reconstruction ability in vivo are significantly improved. The cell will significantly expand the use of existing umbilical cord blood and bone marrow donor banks, reduce the need to recruit a large number of donors to match receptors and increase the probability of HLA matching donors, thereby expanding the infusion of clinical-grade allogeneic cells. The engineered cell, as the source of a universal cell, can achieve an HLA matched off-the-shelf cell therapy and has an important clinical transplant transplantation therapy prospect.

### CROSS REFERENCE

This application claims priority to Chinese Patent Application No. 202410772710.X, first filed with the China National Intellectual Property Administration (CNIPA) on Jun. 17, 2024 and entitled “ENGINEERED CELL AND APPLICATION THEREOF”, which is hereby incorporated herein by reference in its entirety.

### INCORPORATION BY REFERENCE

This application contains a sequence listing XML, which has been submitted electronically and is hereby incorporated by reference in its entirety. Said Sequence Listing XML, created on Jun. 4, 2025, is named “20250530_NZ240044_PO_US_filing_SEQ_LIST.xml”, and is 20,000 bytes in size.

### TECHNICAL FIELD

The present disclosure relates to the field of biotechnology, and in particular, to an engineered cell and application thereof.

### BACKGROUND

Cell infusion can be used to treat tumors, autoimmune diseases and congenital genetic diseases. However, cell infusion between allografts requires strict matching. Cell therapy is limited by the bottleneck problems such as difficulty in finding donors and low success rate of matching. Immune rejection response caused by mismatched human leukocyte antigen (HLA) will lead to the failure of cell infusion. Transplantation failure and graft-versus-host disease (GVHD) are the main obstacles to cell therapy, and seriously affect the therapeutic effect and quality of life of patients. At present, there is no effective solution to the difficulty of cell infusion matching.

The success of allogeneic cell transplantation depends on the matching degree of major histocompatibility complex genes between donors and receptors. Haploid cell infusion can improve the selection of HLA-matched donors. However, due to the immune response of cells and antibodies mediated by HLA molecules, the incidence rate of transplantation failure and GVHD are still obstacles to its successful therapy. Compared with adult donor cells, umbilical cord blood (UCB)-derived cells require less strict HLA matching for transplantation. However, due to the HLA-specific antibody in the receptor, hematopoiesis cannot be completely restored after allogeneic UCB cell infusion; and the occurrence of complications after cell infusion is aggravated by the HLA mismatch degree between UCB donors and receptors.

Therefore, how to regulate the immunogenicity of the infused cells, reduce the rejection of cell infusion and increase the probability of HLA matching donors so as to expand the transplantation of clinical allogeneic cells and improve the implantation and long-term reconstruction of donor cells is an important problem to be solved urgently.

### SUMMARY

### Technical Problem to be Solved

One aspect of the present invention is to provide an engineered cell for the problem in the prior art that cell infusion fails due to the immunogenicity of infused allogenic cells.

Specifically, for the deficiency and actual demand in the prior art, the present disclosure provides a method for lacking or reducing the expression of HLA-A protein and/or HLA-B protein in human cells to obtain a variety of cells with low immunogenicity, thereby solving the above problem.

### Technical Solutions Provided as Follows

An engineered cell, where compared with a wild type cell, the engineered cell lacks or reduces the expression of HLA-A protein and HLA-B protein, and the cell is a human hematopoietic progenitor cell (HSPC).

In some embodiments of the present disclosure, the lack or reduction is implemented by knocking out or knocking down the HLA-A gene and/or an HLA-B gene in the engineered cell.

In the present disclosure, the gene can be knocked out by any suitable method in the prior art. In some embodiments of the present disclosure, the engineered cell includes a gene editing system editing an HLA-A gene and/or an HLA-B gene. The gene is knocked out by the gene editing system.

Further, in some embodiments of the present disclosure, the gene editing system includes transcription activator-like effector nucleases (TALENs), zinc finger nucleases (ZFNs) or a Cas9 protease.

Further, in some embodiments of the present disclosure, the gene editing system includes a Cas9 protease and a guide RNA, where the Cas9 protease is exogenously transferred or expressed in the cell.

Further, in some embodiments of the present disclosure, the guide RNA includes one or more nucleotide sequences shown in SEQ ID Nos.1-21. The sgRNA shown in SEQ ID No.1-21 is used to efficiently knock out or knock down the HLA-A gene. In some embodiments of the present disclosure, the guide RNA further includes the nucleotide sequence shown in SEQ ID No.22, and the nucleotide sequence shown in SEQ ID No.22 is used to efficiently knock out or knock down the HLA-B gene.

Further, in some embodiments of the present disclosure, the guide RNA is selected from one or more of the nucleotide sequence shown in SEQ ID No.1, SEQ ID No.2 or SEQ ID No.3.

In the present disclosure, the gene can be knocked down by any suitable method in the prior art. In some embodiments of the present disclosure, the engineered cell includes an inhibitory nucleic acid targeting the HLA-A and/or HLA-B gene or mRNA.

Further, in some embodiments of the present disclosure, the inhibitory nucleic acid includes an antisense oligonucleotide for the HLA-A and/or HLA-B gene or Protein, a small interfering RNA (siRNA) or a dsRNA used in RNA interference (RNAi).

Another aspect of the present disclosure is to provide application of the engineered cell to preparation of a product for cell infusion.

Another aspect of the present disclosure is to provide a drug composition. The pharmaceutical composition includes the engineered cell, the gene editing system or the inhibitory nucleic acid.

### Beneficial Effects

The engineered cell provided by the present disclosure can significantly reduce the activation of allogeneic T cells without activating NK cells, thus reducing the immune rejection response of the infusion. In addition, the implantation rate and long-term hematopoietic reconstruction ability in vivo of the engineered cell are significantly improved. The cell will significantly expand the use of existing umbilical cord blood and bone marrow donor banks, reduce the need to recruit a large number of donors to match receptors and increase the probability of HLA matching donors, thereby expanding the infusion of clinical-grade allogeneic cells. The engineered cell, as the source of a universal cell, can achieve an HLA matched off-the-shelf cell therapy and has an important clinical therapy prospect.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow result diagram of knock-out/knock-down efficiency of cells in a Ctrl group and each group after HLA-A, HLA-B and HLA-C are respectively knocked out/knocked down according to an embodiment of the present disclosure;

FIG. 2 is a statistical result diagram of editing efficiency of a Ctrl group, an HLA-A-group, an HLA-B-group, an HLA-C-group and an HLA-AB-group according to an embodiment of the present disclosure;

FIG. 3 is a statistical result diagram of efficiency of knocking out of a Ctrl group, an HLA-A-group, an HLA-B-group, an HLA-C-group and an HLA-AB-group according to an embodiment of the present disclosure;

FIG. 4 is a flow result diagram of a Ctrl group, an HLA-A-group, an HLA-B-group, an HLA-C-group and an HLA-AB-group escaping from allogeneic T cell immune response according to an embodiment of the present disclosure;

FIG. 5 is a statistical result diagram of a Ctrl group, an HLA-A-group, an HLA-B-group, an HLA-C-group and an HLA-AB-group escaping from allogeneic T cell immune response according to an embodiment of the present disclosure;

FIG. 6 is a statistical result diagram of a Ctrl group, an HLA-AB-group, an HLA-ABC-group and an HLA-B2M-group activating NK according to an embodiment of the present disclosure;

FIG. 7 is a statistical result diagram of the proportion of human CD45⁺ cells in peripheral blood of mice infused with cells in a Ctrl group, an HLA-A-group, an HLA-B-group and an HLA-AB-group according to an embodiment of the present disclosure;

FIG. 8 is a statistical result diagram of the proportion of each line of cells in peripheral blood of mice infused with cells in a Ctrl group, an HLA-A-group, an HLA-B-group and an HLA-AB-group according to an embodiment of the present disclosure;

FIG. 9 is a statistical result diagram of the implantation rate of human CD45⁺ cells in the bone marrow of mice infused with cells in a Ctrl group, an HLA-A-group, an HLA-B-group and an HLA-AB-group according to an embodiment of the present disclosure;

FIG. 10 is a statistical result diagram of the proportion of each line of cells in the bone marrow of mice infused with cells in a Ctrl group, an HLA-A-group, an HLA-B-group and an HLA-AB-group according to an embodiment of the present disclosure;

FIG. 11 is a statistical result diagram of the proportion of human CD34⁺CD38⁻ cells in the bone marrow of mice infused with cells in a Ctrl group, an HLA-A-group, an HLA-B-group and an HLA-AB-group according to an embodiment of the present disclosure;

FIG. 12 is a statistical result diagram of the proportion of CD34⁺CD38⁺ cells in the bone marrow of mice infused with cells in a Ctrl group, an HLA-A-group, an HLA-B-group and an HLA-AB-group according to an embodiment of the present disclosure;

FIG. 13 is a result diagram of the knock-out effect of 21 sgRNAs detected and screened by a transient transfection method on a 293T cell line according to an embodiment of the present disclosure, where the figure shows the proportion of the cells with HLA-A low expression after knock-out of each sgRNA, the proportion of the HLA-A knock-out cells in the HLA-A-ex2-g13 group with the best knock-out effect can reach 60%, and in addition, the HLA-A gene site targeted by each sgRNA is marked in the figure, P is Promoter, Ex is exon, and Int is Intron;

FIG. 14 is a result diagram of knock-out sites HLA-A-ex2-g13 (SEQ ID No.1), HLA-A-ex3-g167 (SEQ ID No.2) and HLA-A-ex2-g452 (SEQ ID No.3) on a human HLA-A gene according to an embodiment of the present disclosure;

FIG. 15 is a result diagram of further detecting the knock-out effect of three sgRNAs: HLA-A-ex2-g13 (SEQ ID No.1), HLA-A-ex3-g167 (SEQ ID No.2) and HLA-A-ex2-g452 (SEQ ID No.3) with the best HLA-A protein knock-out effect on a 293T cell line on hematopoietic cells derived from umbilical cord blood according to an embodiment of the present disclosure, where the flow diagram shows a gating strategy of CD34⁺, CD34⁺CD90⁺ and CD34⁺CD90⁻ cells;

FIG. 16 and FIG. 17 show the knock-out effect of HLA-A-ex2-g13 (SEQ ID No.1), HLA-A-ex3-g167 (SEQ ID No.2) and HLA-A-ex2-g452 (SEQ ID No.3) on hematopoietic cells derived from umbilical cord blood according to an embodiment of the present disclosure, where FIG. 16 shows that three sgRNAs can effectively knock out HLA-A protein on the surfaces of CD34⁺, CD34⁺CD90⁺ and CD34⁺CD90⁻ cells, the proportions of the CD34⁺CD90⁺ cells knocked out by the three sgRNAs are 94% or above, the proportion of the HLA-A knock-out cells on CD34⁺CD90⁺ in the HLA-A-ex2-g13 (SEQ ID No.1) group can reach 97%, and FIG. 17 shows that the proportion of the HLA-A positive cell population after knock-out of the three sgRNAs is significantly reduced through statistical analysis;

FIG. 18 and FIG. 19 are specificity result diagrams according to an embodiment of the present disclosure, where FIG. 18 shows that compared with a control group, there is no significant difference in the expression of HLA-ABC on the surfaces of the CD34⁺CD90⁺ cells after HLA-A is knocked out by three sgRNAs, indicating that the three sgRNAs have significant specificity for knocking out HLA-A in a targeted manner, the expression of HLA-B and HLA-C molecules on the surfaces of CD34⁺, CD34⁺CD90⁺ and CD34⁺CD90⁻ cells are not affected after HLA-A is knocked out by the three sgRNAs, and FIG. 19 shows that the proportion of the HLA-ABC positive cell population does not change significantly after knock-out of the three sgRNAs through statistical analysis; and

FIG. 20 and FIG. 21 are specificity result diagrams according to an embodiment of the present disclosure, where FIG. 20 shows that compared with a control group, there is no significant difference in the expression of HLA-DR molecules on the surfaces of CD34⁺CD90⁺ cells after HLA-A is knocked out by three sgRNAs, and FIG. 21 shows that the proportion of the HLA-DR positive cell population does not change significantly after knock-out of the three sgRNAs through statistical analysis, indicating that the sgRNA has significant specificity of knocking out HLA-A in a targeted matter, and the expression of HLA-II molecules on the surfaces of CD34⁺, CD34⁺CD90⁺ and CD34⁺CD90⁻ cells are not affected after the HLA-A is knocked out by the three sgRNA.

### SEQUENCE DESCRIPTION

Sequence Table

| Sequence | Sequence | |
|---|---|---|
| Number | Name | Sequence |
| SEQ ID | HLA-A- | GGATGTGAAGAAATACCTCA |
| No. 1 | ex2-g13 | |
| SEQ ID | HLA-A- | GGACCTGCGCTCTTGGACCG |
| No. 2 | ex3-g167 | |
| SEQ ID | HLA-A- | GGATGGAGCCGCGGGCGCCG |
| No. 3 | ex2-g452 | |
| SEQ ID | HLA-A- | GGCCGCCTCCCACTTGCGCT |
| No. 4 | ex3-g448 | |
| SEQ ID | HLA-A- | CTGGTTGTAGTAGCCGCGCA |
| No. 5 | ex2-g274 | |
| SEQ ID | HLA-A- | CGTGTCCCGGCCCGGCCGCG |
| No. 6 | ex2-g440 | |
| SEQ ID | HLA-A- | CACAGCCGCCCACTTCTGGA |
| No. 7 | ex4-g107 | |
| SEQ ID | HLA-A- | ATTGCTGGCCTGGTTCTCCT |
| No. 8 | ex5-g410 | |
| SEQ ID | HLA-A- | ACCCAATGGGAGTGAGAACT |
| No. 9 | promoter- | |
| | g258 | |
| SEQ ID | HLA-A- | GTCCTCCCCATCCCGCTGCC |
| No. 10 | ex4-g461 | |
| SEQ ID | HLA-A- | GAGCCAGAGGATGGAGCCGC |
| No. 11 | ex2-83 | |
| SEQ ID | HLA-A- | ACCCAGTTCTCACTCCCATT |
| No. 12 | promoter- | |
| | 190 | |
| SEQ ID | HLA-A- | GAGAGGGAGAAAAGAAACTG |
| No. 13 | promoter- | |
| | 57 | |
| SEQ ID | HLA-A- | AGGTCAGTGTGATCTCCGCA |
| No. 14 | ex4-g227 | |
| SEQ ID | HLA-A- | GACCCCGCACTCACCCGCCC |
| No. 15 | ex1-g269 | |
| SEQ ID | HLA-A- | CTGGAAACCCGACACCCAAT |
| No. 16 | promoter- | |
| | 28 | |
| SEQ ID | HLA-A- | GAGGGTTCGGGGCGCCATGA |
| No. 17 | ex1-g35 | |
| SEQ ID | HLA-A- | TCTGGAAACCCGACACCCAA |
| No. 18 | promoter- | |
| | g63 | |
| SEQ ID | HLA-A- | GGGGGACTGGGCTGACCGCG |
| No. 19 | int2-g404 | |
| SEQ ID | HLA-A- | CGGCTCCATCCTCTGGCTCG |
| No. 20 | ex2-g246 | |
| SEQ ID | HLA-A- | GGGAGAATCTGAGTCCCGGT |
| No. 21 | promoter- | |
| | g118 | |
| SEQ ID | HLA-B- | CGACGCCGCGAGTCCGAGAG |
| No. 22 | sgRNA | |

### DETAILED DESCRIPTION OF THE EMBODIMENTS

The present invention discloses an engineered cell and application thereof. Those skilled in the art can learn from the contents of the specification to appropriately improve process parameters. It should be particularly noted that all similar substitutions and modifications are apparent to those skilled in the art and are considered to be included within the present invention; and related persons can obviously make a modification or a proper change and combination on the content of this paper without departing from the content, the spirit and the scope of the present invention, so as to implement and apply the technology of the present invention.

In the present invention, unless otherwise specified, scientific and technical terms used herein have the meaning as commonly understood by those skilled in the art. Unless otherwise expressly stated, throughout the specification and claims, the term “including” or its variations such as “including” or “comprising” will be understood as including the stated elements or components, but not excluding other elements or components. The terms “one” (“a”, “an” and “the”) include plural referents. The term “a plurality of” refers to two or more. The terms “such as”, “for example” and the like are intended to refer to exemplary embodiments and are not intended to limit the scope of the present disclosure.

In the present disclosure, when the value range is provided, it should be understood that, unless the context clearly indicates otherwise, the range includes endpoints, and each intermediate value between the upper and lower limits of the range and any other specified value or intermediate value within the specified range and any value within a smaller range between the specified values are covered.

In the present disclosure, the term “about” generally refers to the range of 0.5%-10% above or below the specified value, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below the specified value.

In the present disclosure, unless otherwise specified, scientific and technical terms used herein have the meanings commonly understood by those skilled in the art. The definitions of common terms in molecular biology can be referenced to Lewin's GENES, Twelfth Edition, Jocelyn E. Krebs, Elliott S. Goldstein, Stephen T. Kilpatrick, Press: Jones & Bartlett Learning. The definitions of common terms in biochemistry can be referenced to Lehninger Principles of Biochemistry, Eighth Edition, David L. Nelson, Michael M. Cox, Press: W. H. Freeman. The definitions of common terms in cell biology can be referenced to Molecular Biology of the Cell, Sixth Edition, Bruce Alberts, Alexander Johnson, Julian Lewis, David Morgan, Martin Raff, Keith Roberts, Peter Walter, Press: Garland Science. The definitions of common terms in genetics can be referenced to Genetics: Analysis of Genes and Genomes, Eighth Edition, Daniel L. Hartl, Maryellen Ruvolo, Press: Jones & Bartlett Learning.

Unless otherwise specified, the experimental technologies in the specification adopt the conventional technologies of immunology, biochemistry, chemistry, molecular biology, microbiology, cell biology, genomics and recombinant DNA, which can be referenced to the following standard books: Molecular Cloning: A Laboratory Manual, Cell Biology: A Laboratory Handbook, and the like.

### Terms

the term “human hematopoietic progenitor cell (HSPC)” used in the present disclosure refers to hematopoietic stem cells (HSCs) and/or hematopoietic progenitor cells (HPCs). The hematopoietic stem cells are adult stem cells in the blood system, which have long-term self-renewal ability and the potential to differentiate into various mature blood cells. The various mature blood cells include red blood cells and white blood cells. The hematopoietic progenitor cells are progenitor cells of hematopoietic stem cells that proliferate and differentiate into various blood cells under the regulation of certain microenvironments and certain factors. The hematopoietic progenitor cells are also quite primitive cells with proliferative ability, but have lost the ability of multidirectional differentiation and can only proliferate and differentiate directionally to one or several blood cell lines, so the hematopoietic progenitor cells are also called committed stem cells. In some embodiments of the present disclosure, the hematopoietic progenitor cells can be derived from bone marrow, peripheral blood or umbilical cord blood.

The term “wild type” used in the present disclosure is a concept relative to mutant type, and generally refers to a non-artificially mutagenic individual obtained from nature in research. This individual carries a wild-type genome. In some embodiments of the present disclosure, wild type refers to non-engineered.

The term “reduction” used in the present disclosure means that the expression level (expression quantity) of target protein in cells is smaller than that of target protein in wild type cells. The reduction can be a reduction to about 99%, about 98%, about 95%, about 90%, about 85%, about 80%, about 75%, about 70%, about 65%, about 60%, about 55%, about 50% or less, but not 0%. A method for measuring the expression level (expression quantity) of the target protein may include, for example, Western blot, enzyme-linked immunosorbent assay (ELISA), mass spectrometry, flow cytometry and the like.

The term “knock-out” used in the present disclosure is used to describe the process of completely or partially inactivating a specific gene in an organism by a gene editing technology. The knock-out technology usually involves the use of a gene editing tool, such as a CRISPR-Cas9 system, to accurately position and cut off the DNA sequence of a target gene. After the DNA sequence is cut off, a cell usually tries to repair the broken DNA through a non-homologous end joining (NHEJ) or homologous recombination (HDR) mechanism. However, these repair processes may lead to the insertion, deletion or substitution of gene sequences, thus making the target gene lose the original function or expression level.

The term “knock-down” used in the present disclosure refers to reducing the expression level of a certain gene in an organism by a specific technical means, rather than completely eliminating the function of the gene, for example, implemented through antisense RNA, ribonuclease or interference (CRISPRi) of the CRISPR-Cas system. The main difference between knock-down and knock-out is the degree: knock-out refers to completely or partially eliminating the function of the gene, while knock-down refers to reducing the expression level of the gene. Therefore, knock-down allows researchers to research the influence of the reduced expression quantity of the gene is reduced, rather than the influence of the complete deletion.

The term “HLA-A” used in the present disclosure is the abbreviation of human leukocyte antigen A, also known as MHC class I antigen. It is a glycoprotein expressed on the surfaces of almost all cells in the human body, belonging to a part of major histocompatibility complex (MHC). The heavy chain of the HLA-A molecule is about 45 kDa, and the gene thereof includes 8 exons. The exon 1 encodes a lead peptide, the exons 2 and 3 encode α1 and α2 domains (the two bind to peptide), the exon 4 encodes an α3 domain, the exon 5 encodes a transmembrane region, and the exons 6 and 7 encode a cytoplasmic tail. In particular, the polymorphisms in the exon 2 and the exon 3 are related to the peptide binding specificity of each HLA-A molecule, and the typing of these polymorphisms is usually used for the matching of bone marrow and kidney transplantation. In some embodiments of the present disclosure, the lack or reduction of the expression of HLA-A protein further includes the lack or reduction of the expression of alleles corresponding to the HLA-A protein, including HLA-A1, HLA-A2, HLA-A3, HLA-A11 or HLA-A24.

### CRISPR-Cas System:

The CRISPR-Cas system widely exists in chromosomes of bacteria and archaea, which is related to its immunity. The CRISPR-Cas system is used to defend against foreign genetic materials and gain the ability to resist phages. The CRISPR-Cas system is a third generation gene editing technology after the introduction of ZFN, TALENs and other gene editing technologies. The CRISPR-Cas system can be divided into three main types: Type I, Type II and Type III. The CRISPR Type I system is the earliest discovered and researched CRISPR system, which mainly exists in most bacteria and archaea. The system includes a plurality of Cas proteins and a plurality of CRISPR RNAs (crRNA), forming a complicated Cas protein complex. The complex recognizes and shears a target fragment through the complementary pairing of crRNA and exogenous DNA. The Type I system is characterized by having a plurality of Cas proteins. The Cas3 protein has RNA-dependent DNA nuclease activity and is responsible for shearing the target DNA. The CRISPR Type II system, also known as a CRISPR/Cas9 system, is a gene editing tool most widely used at present. The CRISPR/Cas9 system only requires Cas9 protein and trans-activating CRISPR-derived RNA (trans-activating crRNA, tracrRNA) to form a complex, so that a target DNA fragment can be recognized and sheared. The Cas9 protein has two nuclease activities of HNH and RuvC, which can respectively shear two chains of the target DNA. The CRISPR Type III system has the structure and function similar to those of the Type I system, but has more Cas proteins and a more complicated mechanism. Similar to the Type I system, the Type III system also requires a plurality of Cas proteins and crRNAs to form a complex, and recognizes and shears a target fragment through the complementary pairing with the exogenous DNA. However, the shearing mechanism of the Type III system is different from that of the Type I system, which depends on the RNA enzyme activity of the Cas protein to cut the target DNA. In the embodiments of the present disclosure, the sgRNA is used for the Type II CRISPR-Cas system.

### Cas Protein:

In the embodiments of the present disclosure, the Cas protein used by the CRISPR-Cas system is mainly Cas9. Cas9 is an endonuclease. Under the guidance of the sgRNA (small guide RNA), Cas9 can accurately identify and cut DNA sequences. In some embodiments of the present disclosure, Cas9 protein can be isolated or loaded on a vector for intracellular expression. In addition to Cas9, in some other embodiments of the present disclosure, other accessory proteins may be included, including, but not limited to, Cas1, Cas2 and the like. The accessory proteins may play an auxiliary role in the DNA cutting process, or participate in other links of the CRISPR system, such as DNA acquisition and processing.

In the embodiments of the present disclosure, Cas protein may be modified. The modification may include mutation, insertion or deletion of amino acid residues, codon optimization, and some other chemical modifications. The objective may be to change the activity or specificity of Cas protein without changing the main function of Cas protein. For example, the US patent US20180100148A1 describes the mutation of nucleic acid and protein of Streptococcus pyogenes Cas9 to reduce the off-target effect of the system.

### Inhibitory Nucleic Acid:

In the present disclosure, the term “inhibitory nucleic acid (INA)” is a kind of nucleic acid molecules that can inhibit the expression of a specific gene or protein function. The nucleic acid molecules can combined with a complementary sequence of a target gene or protein through specific sequence design and modification, thereby interfering with the normal function or expression level. The common examples of the inhibitory nucleic acid include antisense oligonucleotide, small interfering RNA(siRNA), RNA interference (RNAi) and the like.

### Guide RNA and Combination Thereof

In the embodiments of the present disclosure, the nucleotide sequence of the sgRNA shown in SEQ ID Nos.1-21 can efficiently knock down and/or knock out a human HLA-A gene. The nucleotide sequence shown in SEQ ID No.22 is used to efficiently knock out or knock down the HLA-B gene. After the nucleotide sequence is known, it can be obtained by suitable methods in the prior art, including but not limited to chemical synthesis, polymerase chain reaction (PCR) and the like.

In the embodiments of the present disclosure, by the CRISPR gene editing system, the sgRNA is used to recognize and knock down and/or knock out the human HLA-A gene in a cell, the efficiency of the sgRNA in a 293T cell line can reach 20% or above, and the highest efficiency on cord blood cells derived from umbilical cord blood can reach 97%, which is far superior to the method in the prior art. The efficiencies of three sgRNAs such as HLA-A-ex2-g13, HLA-A-ex3-g167 and HLA-A-ex2-g452 are the best. Therefore, in some embodiments of the present disclosure, the nucleotide sequence of the sgRNA is shown in SEQ ID No.1, SEQ ID No.2 or SEQ ID No.3.

In the gene editing process, the sgRNA can be used alone, and in some embodiments of the present disclosure, about 20%, about 30%, about 40%, about 50%, about 60%, about 65%, about 70%, about 75%, about 80%, about 85%, about 90%, about 95%, about 98% and about 99% of editing efficiency can be achieved; and the sgRNA can be used in combination, or other sgRNAs can be added, and in some other embodiments of the present disclosure, about 20%, about 30%, about 40%, about 50%, about 60%, about 65%, about 70%, about 75%, about 80%, about 85%, about 90%, about 95%, about 98% and about 99% of editing efficiency can also be achieved. In some embodiments of the present disclosure, the sgRNA can be loaded in the same container, or can be loaded in different containers and transferred into cells.

The nucleotide sequence of the above sgRNA can be modified. The modification includes, but is not limited to, substitution, insertion, deletion, exchange or replacement regions of one or more nucleotides for improvement, and modifications on the nucleotide sequence by other groups. In some embodiments of the present disclosure, the mutated sgRNA nucleotide sequence may have at least 20%, at least 30%, at least 40%, at least 50%, at least 60%, or at least 70%, at least 80%, at least 85%, at least about 90%, at least about 95%, at least about 96%, at least about 97%, at least about 98% or at least bout 99% of identity compared with SEQ No. 1-21.

To make those skilled in the art better understand the technical solutions of the present invention, the present invention will be further described in detail below in combination with the specific embodiments.

### EMBODIMENTS

### Embodiment 1: Construction of Cells with HLA-A, HLA-B, HLA-C and HLA-AB Cells Knocked Out/Knocked Down

1. CD34⁺ cells derived from umbilical cord blood, bone marrow or peripheral blood were divided into five groups, namely a Ctrl group, an HLA-A knocked out/knocked down group, an HLA-B knocked out/knocked down group, an HLA-C knocked out/knocked down group and an HLA-AB knocked out/knocked down group. Cells in each group were resuspended with electrotransfection buffer, RNP not including sgRNA was added into the Ctrl group, RNP including HLA-A sgRNA was added into the HLA-A knocked out group, RNP including HLA-B sgRNA was added into the HLA-B knocked out group, RNP including HLA-C sgRNA was added into the HLA-C knocked out group, and RNP including HLA-A sgRNA and HLA-B sgRNA were added into the HLA-AB group.

2. The cells added with RNP in each group were subjected to electrotransfection in an electrotransfection instruction. A gene editing method is as follows:

(1) Protein and sgRNA (1:2) in a lonza electrotransfection kit were put into a 1.5 ml EP tube for reaction at room temperature for 10-15 minutes.

(2) The cells were counted and were put into a 50 ml centrifugal tube after cell suspension was absorbed. 500 g was centrifuged for 5 minutes.

(3) The supernatant was discarded, each tube was washed once with PBS resuspended cells, and 500 g was centrifuged for 5 minutes.

(4) The supernatant was discarded, the cells were resuspended with buffer, RNP (system with the final volume of 20 ul) prepared in step (1) was added, blowing, beating and uniform mixing were performed with a gun tip, and the mixture was put into an electrotransfection pore plate.

(5) After electrotransfection by the lonza electrotransfection instrument, the cell suspension on the electrotransfection pore plate was absorbed into a 15 ml centrifugal tube. 500 g was centrifuged for 5 minutes and the supernatant was discarded.

(6) Each tube is resuspended with a 2 ml culture medium and then transferred into a culture dish.

3. After electrotransfection, the cells were resuspended with a culture medium rapidly and were cultured in an incubator for 24 hours.

4. For the efficiency of sequencing, detecting and knocking out gene fragments, the editing efficiency of each gene was detected by flow cytometry.

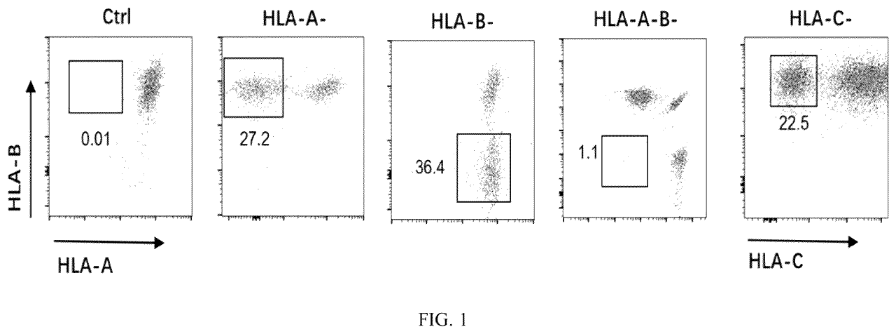

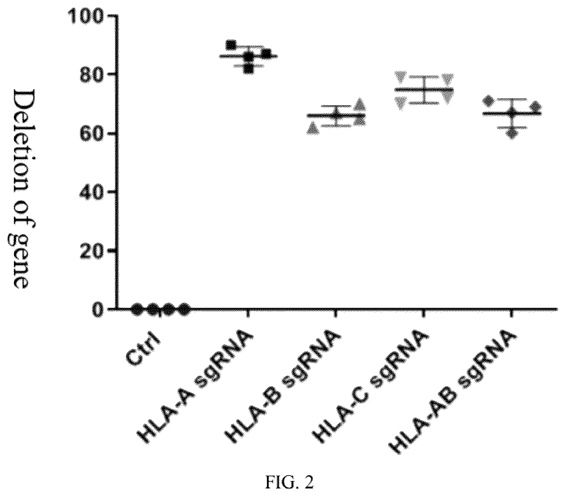

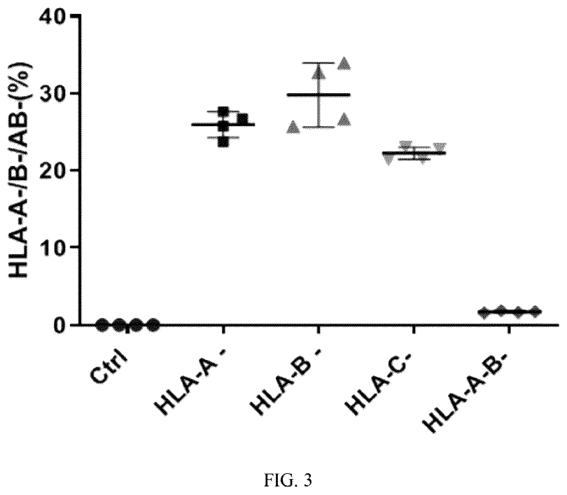

The results are shown in FIG. 1, FIG. 2 and FIG. 3, the percentage of the gene editing efficiency of the HLA-A, HLA-B, HLA-C and HLA-AB groups is respectively 86.25±3.30, 66±3.36, 74.75±4.42 and 66.75±4.78; and the percentage of the gene efficiency of knocking out is respectively 25.99±1.67, 29.81±4.15, 22.23±0.79 and 1.71±0.13.

### Embodiment 2: Obtaining Three Engineered Cells with Immunogenicity Reduced by Knocking Out/Knocking Down HLA-A, HLA-B, HLA-B and HLA-AB Genes

1. Cells in the Ctrl group, the HLA-A knocked out/knocked down group, the HLA-B knocked out/knocked down group, the HLA-C knocked out/knocked down group and the HLA-AB knocked out/knocked down group were pretreated with IFN-7 of 50 ng/ml for 48 hours, and sgRNA and RNP were added into each group for electrotransfection (RNP not including sgRNA was added into the Ctrl group).

2. Human peripheral blood cells were separated, including the following method:
(1) the freshly obtained healthy human Peripheral blood mononuclear cell (PBMC) was placed in a sterile 50 mL centrifugal tube, red blood cell lysis solution was added, and after full mixing, standing was performed at room temperature for not less than 6 minutes, and red blood cells were lysed;
(2) centrifugation was performed at 4° C. and 1500 rpm for 5 minutes;
(3) the supernatant was discarded, cells were resuspended with 20 mL of PBE per tube and fully mixed, 10 uL were taken for counting, and centrifugation at 4° C. and 1500 rpm for 5 minutes; and
(4) the supernatant was discarded, and the collected human peripheral cells were resuspended with IMDM-FBS for later use.

3. 500 μL of PBS was added into PBMC for mixing uniformly to prepare single-cell suspension for sorting T cells. Preparation of RPMI 1640 complete culture medium: RPMI 1640 basic culture medium+10% FBS+1% Glutamax+1% penicillin-streptomycin solution. The subsequent operation steps were performed in the dark. Preparation of CFSE working solution: CFSE 1 μL+500 μL PBS in each part was completely blown, beaten and mixed uniformly.

4. One part of CFSE working solution was respectively added into resuspended single-cell suspension for completely blowing, beating and uniformly mixing, and standing was performed at room temperature for 10 minutes.

5. 1 ml of precooled 50% FBS was added respectively and rapidly, blowing, beating and uniform mixing were performed to stop dyeing, standing was performed on ice for 2 minutes, and 300 g was centrifuged for 10 minutes.

6. A 2 ml 1640 complete culture medium was added respectively for blowing, beating and uniform mixing, and 300 g was centrifuged for 10 minutes.

7. Step 6 was repeated.

8. The engineered cells pretreated for 48 hours on the first day and T cells were mixed in a 96-well U-shaped bottom plate at a ratio of 1:1 in RPMI-1640 including glutamine, 10% FBS and 20 U/mL IL-2 for 3-5 days.

9. The percentage of proliferative CD8⁺ T cells (negative T cells labeled by CFSE) was detected by flow cytometry.

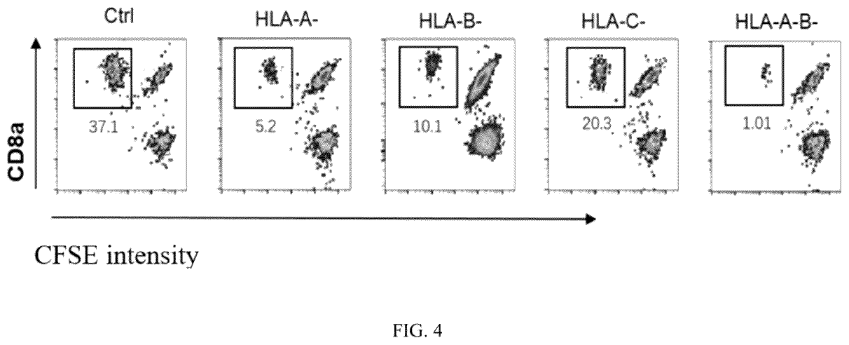

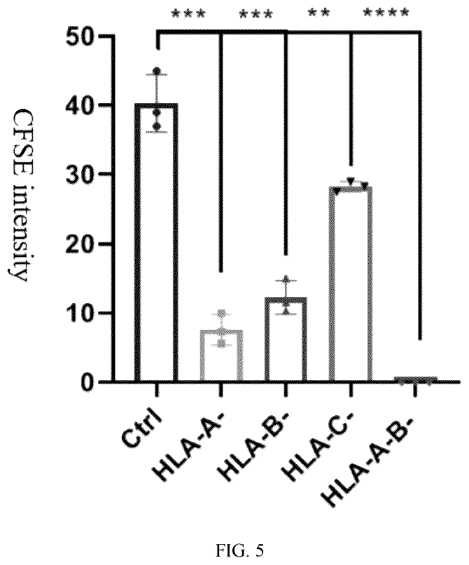

The results are shown in FIG. 4 and FIG. 5. The results indicate that after co-culture of the gene-edited cells and T cells, compared with the Ctrl group, knocking out/knocking down HLA-A, HLA-B and HLA-AB can significantly reduce the activation on allogeneic T cells, but knocking out HLA-C can still significantly activate the T cells.

### Embodiment 3: The Influence of the Cells with HLA-C Knocked Out/Knocked Down on the Activity of NK Cells

The cells with HLA-B2M knocked out/knocked down were set as a positive control group, HLA-A and HLA-B were knocked out/knocked down simultaneously by the method in Embodiment 1 to obtain HLA-AB group cells, and HLA-A, HLA-B and HLA-C were knocked out/knocked down at the same time to obtain HLA-ABC group cells.

2. RNP not including sgRNA was added in the Ctrl group, and the rest were the same as the experimental groups. Cells in each group subjected to co-electrotransfection with RNP were pretreated with IFN-7 of 50 ng/mL for 48 hours.

3. 500 ul of PBS was added into PBMC to mix uniformly and prepare single-cell suspension, and NK cells were sorted by CD3 and CD56 antibodies.

4. Preparation of RPMI 1640 complete culture medium: RPMI 1640 basic culture medium+10% FBS+1% Glutamax+1% penicillin-streptomycin (PS) solution.

5. Cells in the Ctrl group, the HLA-AB group, the HLA-ABC group and the HLA-B2M group and the NK cells were co-cultured in a 96-well plate with a U-shaped bottom for 5 hours. The percentage of the CD107a positive cells of the NK cells was detected by flow cytometry.

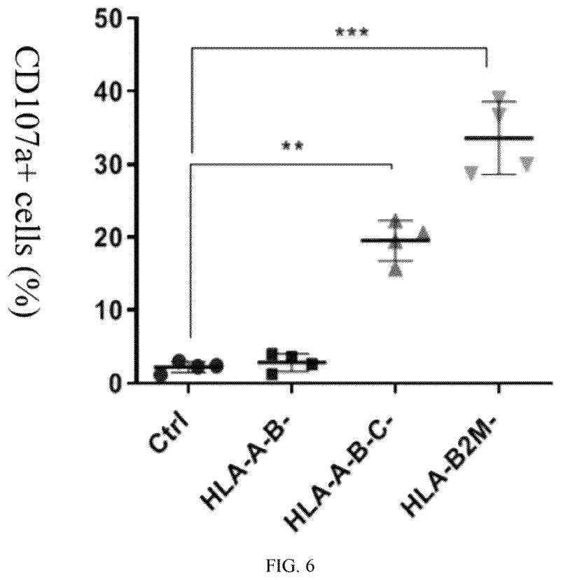

The statistical result is shown in FIG. 6. The result indicates that compared with the Ctrl group, knocking out/knocking down HLA-AB will not activate the NK cells, but knocking out/knocking down HLA-C will significantly activate the NK cells.

### Embodiment 4: The In Vivo Implantation and Reconstruction Abilities of the Engineered Cells are Significantly Enhanced

Three kinds of engineered cells with low immunogenicity HSPC (human CD34⁺ cells with HLA-A, HLA-B and HLA-AB knocked out/knocked down) were respectively transplanted into immunodeficient mice, and the human cell implantation rate and the hematopoietic reconstruction situation in each group were dynamically detected, including the following method:

1. Female NOG mice aged 6-8 weeks and weighing 18-22 g were randomly grouped. Before transplantation, the mice were irradiated with 2Gy X-ray at a dose rate of 1.2 Gy/min.

2. Cells with human HLA-A, HLA-B and HLA-AB knocked out/knocked down were obtained by the method in Embodiment 1 and were transplanted into the NOG mice by caudal veins according to 1×10⁵ cells for each mouse, and the proportion of human CD45⁺ cells in peripheral blood of mice was continuously detected from 4 to 20 weeks after transplantation.

3. 20 weeks after transplantation, the mice were killed by neck removal, and bone marrow cells of the mice were collected, washed with staining buffer, resuspended, added with antibodies and incubated for 30 minutes at 4° C. in the dark.

4. The bone marrow cells were washed once with 2 mL of dyeing buffer, and the human cell implantation in the bone marrow of the mice and the composition of each line were detected by a flow cytometer.

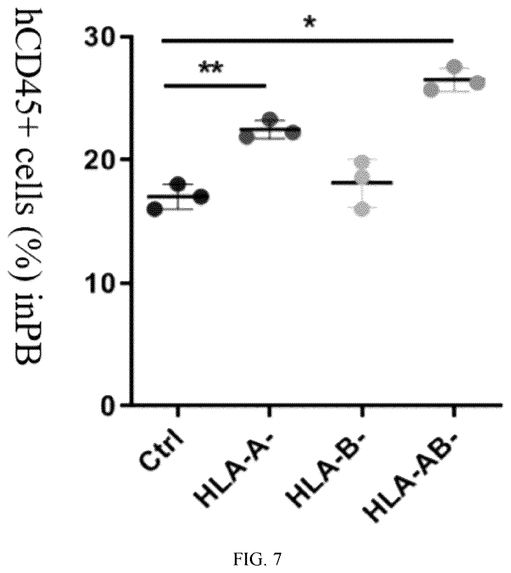

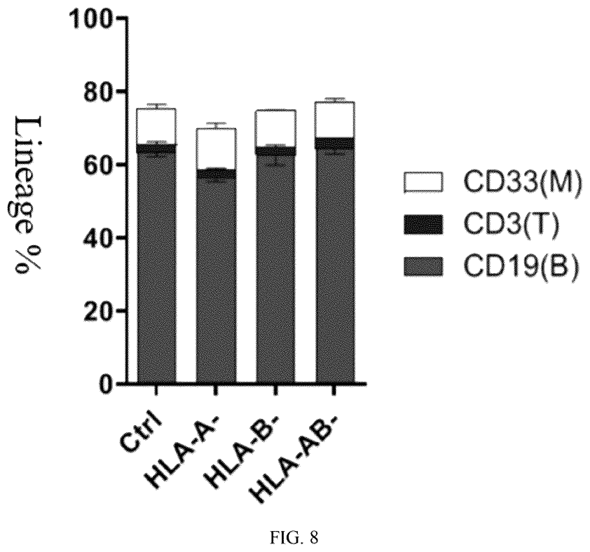

The result shows that compared with the control group, the percentage of the human engineered cells in the CD45⁺ cells in the peripheral blood of the mice are significantly increased, the main cells are CD33⁺ myeloid cells and CD19⁺ B cells, and the results are shown in FIG. 7 and FIG. 8.

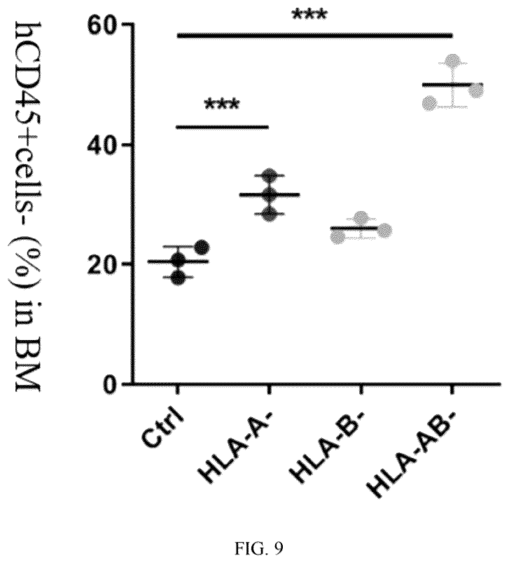

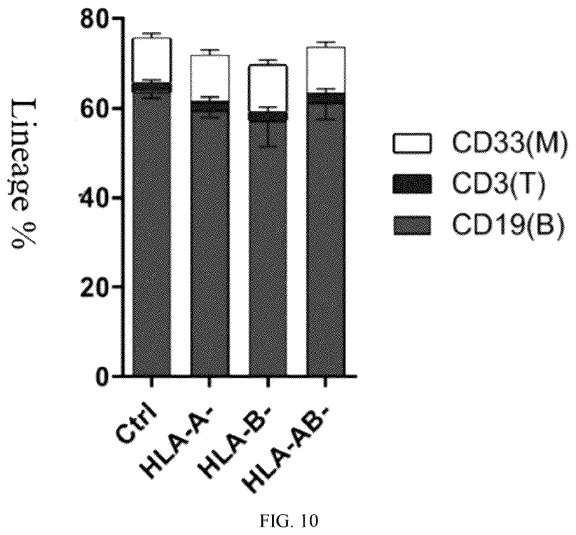

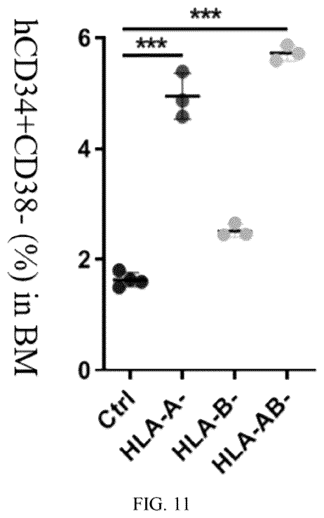

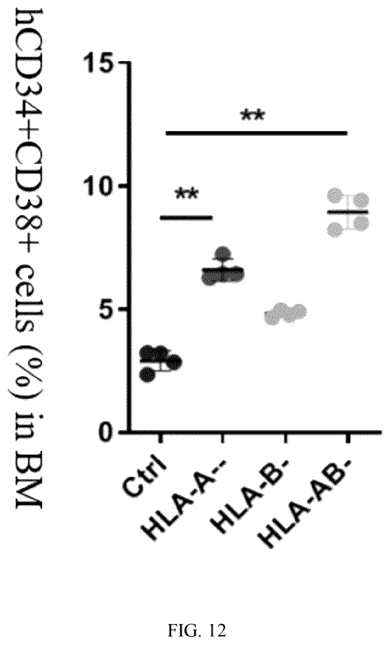

In the bone marrow of the mice, the implantation rate of the human engineered cells is also higher than that of the control group, the main cells are CD33⁺ myeloid cells and CD19⁺ B cells, and the results are shown in FIG. 9 and FIG. 10, indicating that the reconstruction of the engineered cells in each line in vivo is not affected after HLA gene modification. In addition, in the bone marrow of the mice, the proportions of the engineered cells such as CD34⁺CD38 and CD34⁺CD38⁺ cells are significantly higher than those of the control group (the results are shown in FIG. 11 and FIG. 12.)

### Embodiment 5: The Knock-Out Effect of sgRNA was Inspected on the 293T Cell Line by a Transient Transfection Method

1) 293T cells were paved on a 24-well plate 24 hours in advance, with 2×10⁵ cells for each well.

2) The DMEM complete culture medium was replaced with a 0.4 ml DMEM culture medium (including 10% FBS and not including penicillin/streptomycin), and the Penicillin/streptomycin was put into an incubator.

3) A transfection system was prepared: a 50 ul optim culture medium was first added into a 1.5 ml EP tube, target plasmid of 1 ug/well (0.5 ug/well for each of the sg plasmid and the Cas9 plasmid, and the sg plasmid prepared by a conventional method) was added, and inverted mixing was performed without swirling.

4) Another 1.5 ml EP tube was taken, 50 ul optim culture medium was first added, PEI of 3 ug/well was added, and inverted mixing was performed without swirling.

5) The mixture was placed at room temperature for 5 minutes.

6) 3) and 4) were mixed into one tube, inverted mixing was performed without swirling, and standing was performed for 20 minutes.

7) The plasmid system was added into a dish and shaken well.

8) The 0.4 ml DMEM culture medium was replaced with the DMEM complete culture medium after 12-16 hours.

9) 48 hours after transfection, puromycin of 2 μg/ml was added for medicine screening.

10) 48 hours after puromycin medicine screening, the HLA-A knock-out effect of each sgRNA was detected.

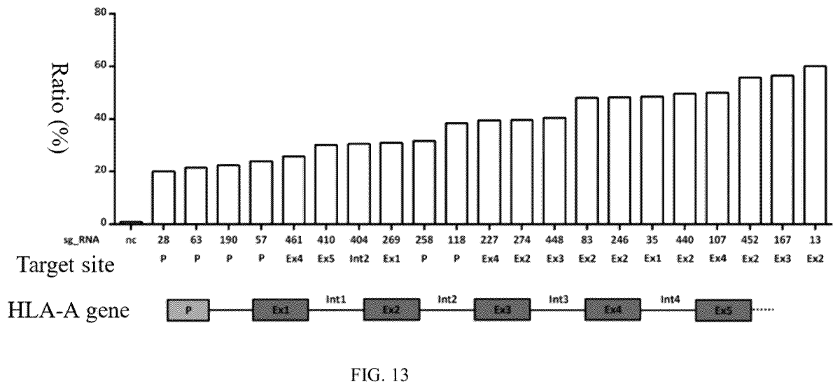

11) The flow detection result shows that in 21 sgRNAs, the knock-out effects of HLA-A-ex2-g13 (SEQ ID No.1), HLA-A-ex3-g167 (SEQ ID No.2) and HLA-A-ex2-g452 (SEQ ID No.3) are better, where the proportion of the HLA-A knock-out cells in the HLA-A-ex2-g13 can reach 60%. (The result is shown in FIG. 13).

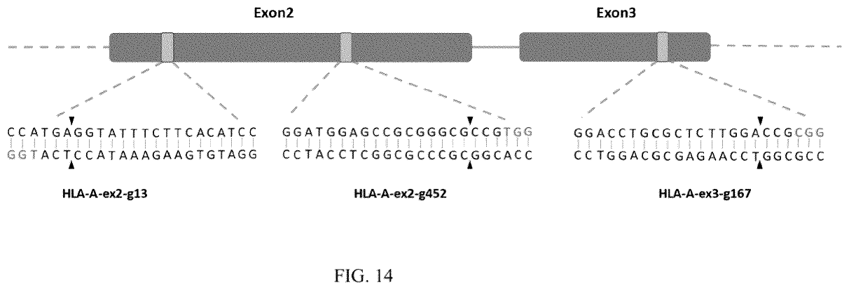

12) The knock-out sites of HLA-A-ex2-g13 (SEQ ID No.1), HLA-A-ex3-g167 (SEQ ID No.2) and HLA-A-ex2-g452 (SEQ ID No.3) on the human HLA-A gene are shown in FIG. 14.

### Embodiment 6: The Knock-Out Effect of sgRNA was Detected on Hematopoietic Cells Derived from Umbilical Cord Blood

1. CD34⁺ cells of umbilical cord blood were enriched, and the cells were put in a hematopoietic cell basic culture medium overnight. The hematopoietic cell basic culture medium is an SFEMII culture medium added with the following cell factors (Table 1):

TABLE 1

| Cell Factor | Concentration |
|---|---|
| hSCF | 100 ng/ml |
| hFlt3-L | 50 ng/ml |
| hTPO | 100 ng/ml |

2. Three sgRNAs with better knock-out effect on the 293T cells were selected: HLA-A-ex2-g13 (SEQ ID No.1), HLA-A-ex3-g167 (SEQ ID No.2) and HLA-A-ex2-g452 (SEQ ID No.3), and the HLA-A protein on the surfaces of CD34⁺ cells were knocked out according to a Lonza P3 Primary Cell 4D-Nucleofector™ X Kit S kit electrotransfection method and through an electrotransfection RNP method.

1) CD34⁺ cells were counted, with 2×10⁵ cells for each sample.

2) An electrotransfection system was prepared: buffer was added in each tube first, and then 75 pmol of spCas9 protein and 150 pmol of sgRNA were added, totaling 5 ul. Reaction was performed at room temperature for 20-30 minutes.

3) Cells were resuspended with 15 ul of buffer, RNP was added (prepared into a 20 ul system), and the system was blown and beaten for more than 10 times. Liquid was added without adding bubbles.

4) Operation was performed according to the instructions of a 4D electrotransfection instrument.

5) After the electrotransfection, cell suspension in an electrotransfection group was transferred to a 1.5 ml centrifugal tube. 500 g was centrifuged for 5 minutes and the supernatant was discarded.

3. The cells were transferred to a 24-well plate for culture and observation. After 72 hours, the expression situations of HLA-A, HLA-ABC and HLA-DR on the surfaces of CD34⁺, CD34⁺CD90⁺ and CD34⁺CD90⁻ cells were respectively detected by a flow technology. The antibodies labeled by the flow technology are shown in Table 2.

TABLE 2

| Detection Protein | Fluorescein | Antibody (ul/sample) |
|---|---|---|
| CD34 | PE | 1 |
| CD90 | percp-cy5.5 | 1 |
| HLA-A2 | APC-cy7 | 0.5 |
| HLA-ABC | APC | 0.5 |
| HLA-DR | FITC | 0.5 |

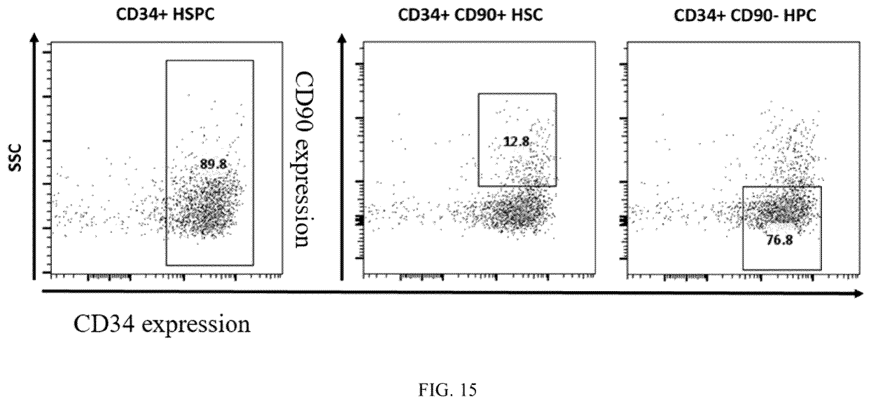

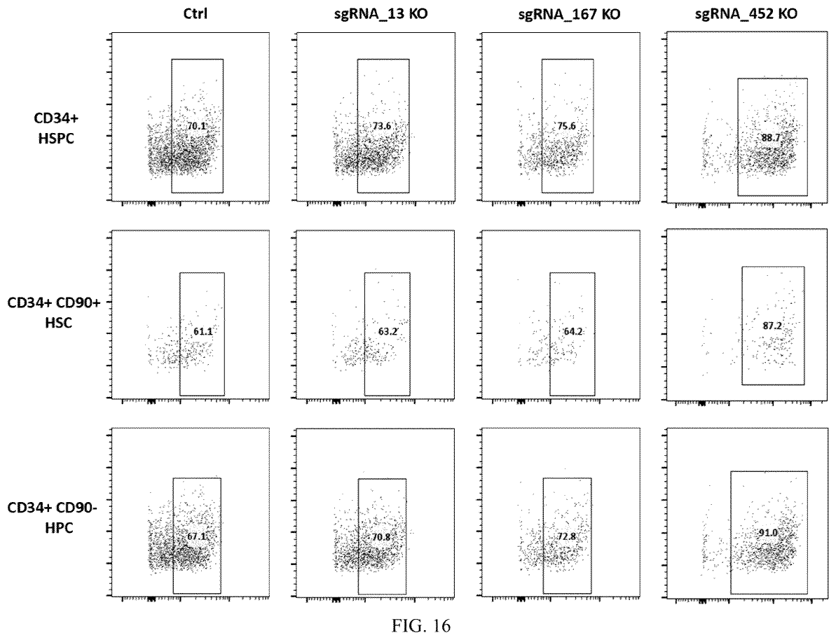

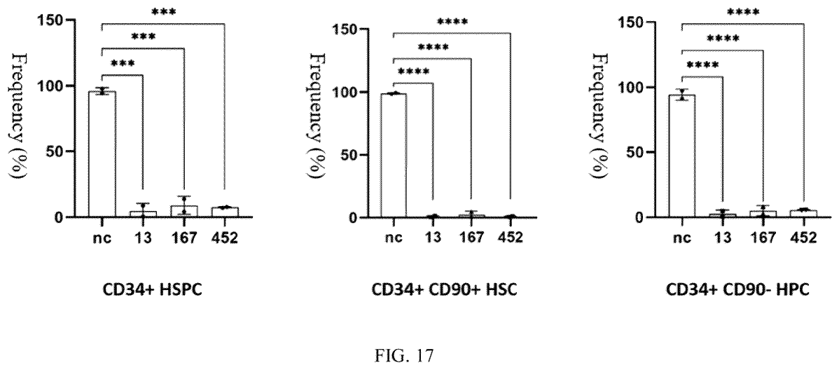

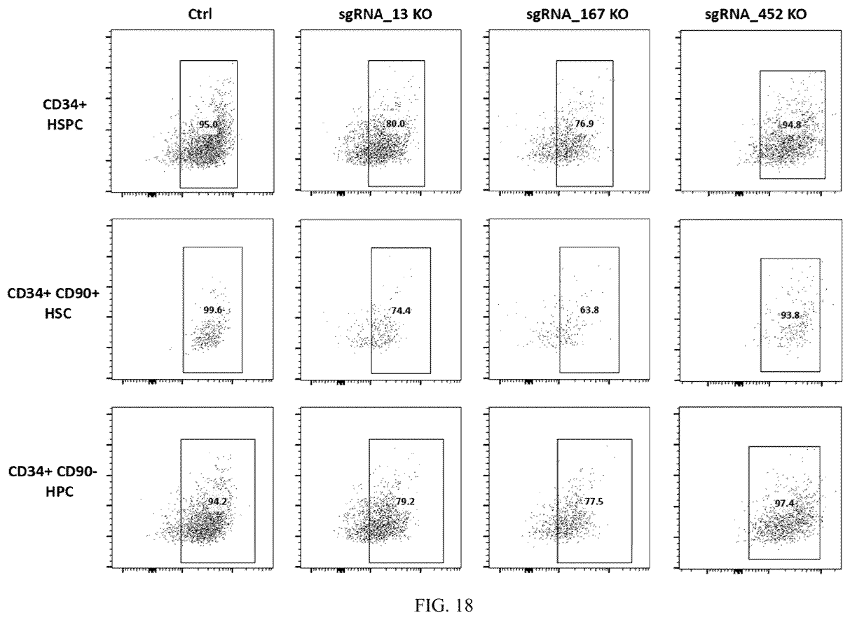

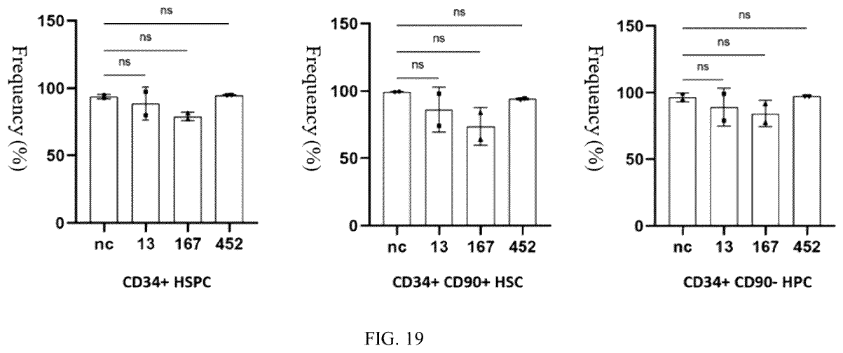

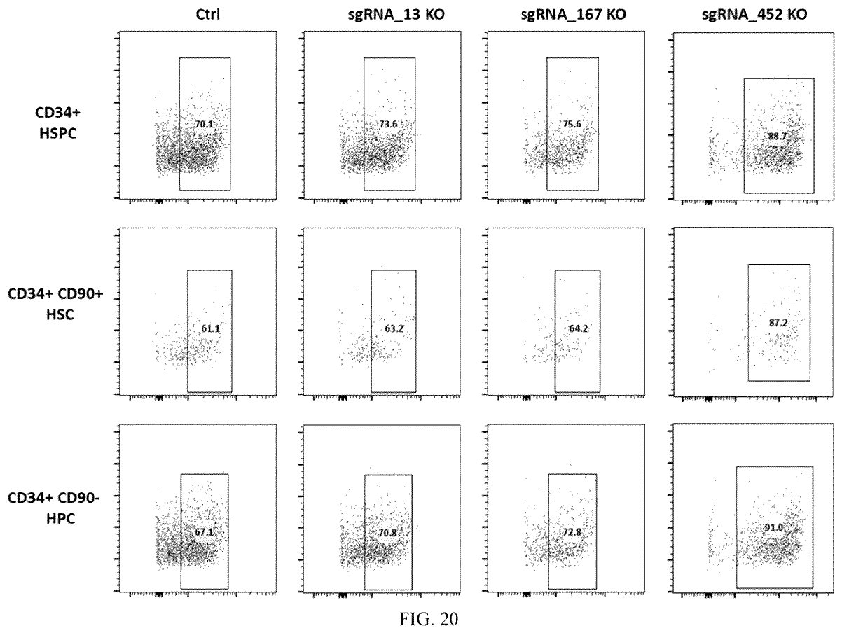

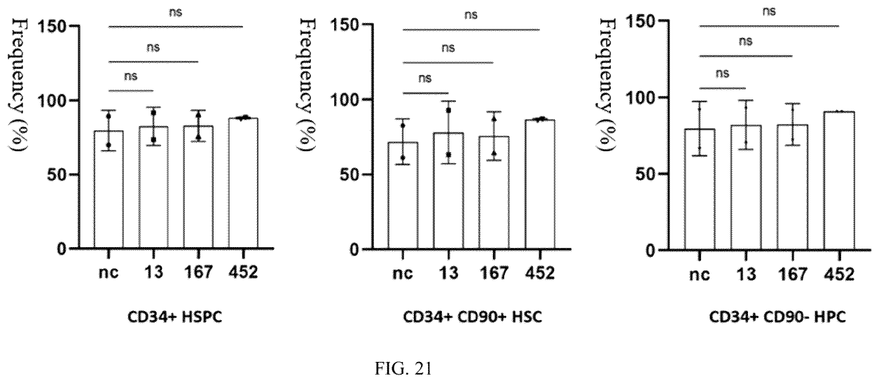

4. The flow results show that three sgRNAs can significantly knock out the HLA-A protein on the surfaces of CD34⁺, CD34⁺CD90⁺ and CD34⁺CD90 cells, the knock-out proportion of the CD34⁺ cells by the three sgRNAs are 94% or above, and the HLA-A knock-out cell proportion on the CD34⁺CD90⁺ cells in the HLA-A-ex2-g13 group can reach 97% (FIG. 15 is a flow gating strategy, and the results are shown in FIG. 16 and FIG. 17). In addition, there is no significant change in the proportion of the HLA-ABC and HLA-DR positive populations on the CD34⁺, CD34⁺CD90⁺ and CD34⁺CD90⁻ cells in the three sgRNA groups. After statistical analysis, there is no significant influence (the results shown in FIG. 20 and FIG. 21) on the expression of HLA-B and HLA-C (the results shown in FIG. 18 and FIG. 19) and HLA-II molecules on three groups of cell HLA-I molecules after the HLA-A is knocked by the three sgRNAs. That is, the sgRNA we used has high targeting.

The above are preferred embodiments of the present invention, and it should be noted that, for those of ordinary skill in the art, several improvements and modifications may be made without departing from the principle of the present invention, and the improvements and modifications are also regarded to be within the protection scope of the present invention.

What is claimed is:

1. An engineered cell, wherein compared with a wild type cell, the engineered cell reduces the expression of HLA-A protein, and the cell is a human hematopoietic progenitor cell (HSPC);
the engineered cell comprises a gene editing system editing an HLA-A gene;
the gene editing system comprises a Cas9 protease and a guide RNA; and
the nucleotide sequence of the guide RNA is shown in SEQ ID No.1.

2. The engineered cell according to claim 1, wherein the reduction is implemented by knocking out or knocking down the HLA-A gene in the engineered cell.


SEQUENCE LISTING (22 sequences)

SEQ ID NO: 1 - RNA, 20 nt, synthetic construct
  source 1..20: mol_type = other RNA
ggatgtgaagaaatacctca

SEQ ID NO: 2 - RNA, 20 nt, synthetic construct
  source 1..20: mol_type = other RNA
ggacctgcgctcttggaccg

SEQ ID NO: 3 - RNA, 20 nt, synthetic construct
  source 1..20: mol_type = other RNA
ggatggagccgcgggcgccg

SEQ ID NO: 4 - RNA, 20 nt, synthetic construct
  source 1..20: mol_type = other RNA
ggccgcctcccacttgcgct

SEQ ID NO: 5 - RNA, 20 nt, synthetic construct
  source 1..20: mol_type = other RNA
ctggttgtagtagccgcgca

SEQ ID NO: 6 - RNA, 20 nt, synthetic construct
  source 1..20: mol_type = other RNA
cgtgtcccggcccggccgcg

SEQ ID NO: 7 - RNA, 20 nt, synthetic construct
  source 1..20: mol_type = other RNA
cacagccgcccacttctgga

SEQ ID NO: 8 - RNA, 20 nt, synthetic construct
  source 1..20: mol_type = other RNA
attgctggcctggttctcct

SEQ ID NO: 9 - RNA, 20 nt, synthetic construct
  source 1..20: mol_type = other RNA
acccaatgggagtgagaact

SEQ ID NO: 10 - RNA, 20 nt, synthetic construct
  source 1..20: mol_type = other RNA
gtcctccccatcccgctgcc

SEQ ID NO: 11 - RNA, 20 nt, synthetic construct
  source 1..20: mol_type = other RNA
gagccagaggatggagccgc

SEQ ID NO: 12 - RNA, 20 nt, synthetic construct
  source 1..20: mol_type = other RNA
acccagttctcactcccatt

SEQ ID NO: 13 - RNA, 20 nt, synthetic construct
  source 1..20: mol_type = other RNA
gagagggagaaaagaaactg

SEQ ID NO: 14 - RNA, 20 nt, synthetic construct
  source 1..20: mol_type = other RNA
aggtcagtgtgatctccgca

SEQ ID NO: 15 - RNA, 20 nt, synthetic construct
  source 1..20: mol_type = other RNA
gaccccgcactcacccgccc

SEQ ID NO: 16 - RNA, 20 nt, synthetic construct
  source 1..20: mol_type = other RNA
ctggaaacccgacacccaat

SEQ ID NO: 17 - RNA, 20 nt, synthetic construct
  source 1..20: mol_type = other RNA
gagggttcggggcgccatga

SEQ ID NO: 18 - RNA, 20 nt, synthetic construct
  source 1..20: mol_type = other RNA
tctggaaacccgacacccaa

SEQ ID NO: 19 - RNA, 20 nt, synthetic construct
  source 1..20: mol_type = other RNA
gggggactgggctgaccgcg

SEQ ID NO: 20 - RNA, 20 nt, synthetic construct
  source 1..20: mol_type = other RNA
cggctccatcctctggctcg

SEQ ID NO: 21 - RNA, 20 nt, synthetic construct
  source 1..20: mol_type = other RNA
gggagaatctgagtcccggt

SEQ ID NO: 22 - RNA, 20 nt, synthetic construct
  source 1..20: mol_type = other RNA
cgacgccgcgagtccgagag